(19) United States
(12) Patent Application Publication
(10) Pub. No.: US 2025/0185975 A1
(43) Pub. Date: Jun. 12, 2025

(54) ESTIMATION OF SERUM POTASSIUM AND/OR GLOMERULAR FILTRATION RATE FROM ELECTROCARDIOGRAM FOR MANAGEMENT OF HEART FAILURE PATIENTS

(71) Applicant: Medtronic, Inc., Minneapolis, MN (US)

(72) Inventors: Abhijit Kadrolkar, St. Paul, MN (US); Jeffrey M. Gillberg, Minneapolis, MN (US); Sierra C. Mulrine, Bordentown, NJ (US)

(21) Appl. No.: 18/707,642

(22) PCT Filed: Nov. 14, 2022
(86) PCT No.: PCT/IB2022/060925

§ 371 Date: May 6, 2024

Related U.S. Application Data
(60) Provisional application No. 63/264,346, filed on Nov. 19, 2021.

(51) Int. Cl. A61B 5/364; A61B 5/00; A61B 5/11; A61B 5/20; A61B 5/352; A61B 5/355; G16H 50/70

(52) U.S. Cl. CPC A61B 5/364; A61B 5/11; A61B 5/201; A61B 5/352; A61B 5/355; A61B 5/4836; A61B 5/7264; A61B 5/746; G16H 50/70; A61B 2562/0219

(57) ABSTRACT

This disclosure is directed to devices, systems, and techniques for estimating a serum potassium level. An example system includes a plurality of electrodes, sensing circuitry configured to sense an ECG of a patient, and processing circuitry. The processing circuitry is configured to determine a T-wave morphology in the ECG and based on the T-wave morphology, determine an estimate of serum potassium in blood of the patient. The processing circuitry is configured to determine that the estimate of serum potassium in the blood satisfies a threshold and based on the estimate of serum potassium in the blood satisfying the threshold, generate an indication for output that is based at least in part on the estimate of serum potassium in the blood satisfying the threshold.

### TECHNICAL FIELD

The disclosure relates generally to medical device systems and, more particularly, medical device systems configured to monitor patient parameters.

### BACKGROUND

Some types of medical devices may be used to monitor one or more physiological parameters of a patient. Such medical devices may include, or may be part of a system that includes, sensors that detect signals associated with such physiological parameters. Values determined based on such signals may be used to assist in detecting changes in patient conditions, in evaluating the efficacy of a therapy, or in generally evaluating patient health.

### SUMMARY

In general, the disclosure is directed to devices, systems, and techniques for using a medical device system to estimate serum potassium in blood of a patient and/or kidney function of a patient. A serum potassium level, kidney function, and blood pressure are biomarkers that heart failure cardiologists utilize to effectively titrate heart failure medications. Kidney function may be determined by serum creatine levels in the blood. For example, Glomerular Filtration Rate (GFR) may be calculated from serum creatinine, to obtain a quantitative measure of kidney function.

Normally, serum potassium levels and serum creating levels are obtained by invasively drawing blood from a patient. However, drawing blood is not an optimal long-term monitoring process, as the patient must repeatedly travel to a medical clinic to have their blood drawn. Additionally, continuous monitoring of serum potassium and/or serum creatine is not feasible through drawing blood.

According to the techniques of this disclosure, a medical device system may monitor an ECG of a patient and estimate the serum potassium in the blood based on a morphology of a T-wave in the ECG and/or estimate a GFR from the ECG. Such techniques may facilitate the remote and/or continuous monitoring of the serum potassium in the blood of the patient and/or kidney function of the patient. In some examples, the morphology of the T-wave may be normalized based on a preceding R-wave morphology, e.g., the R-wave immediately precedes the T-wave. In some examples, the normalized T-wave may be averaged across several successive heart beats, such as over a 30 second period or longer. In some examples, a machine learned patient-specific model and/or a machine learned population averaged model may be used by the medical device system when determining the estimate of the serum potassium in the blood. The medical device system may determine that the serum potassium satisfies a threshold and generate an indication for output based at least in part on the serum potassium satisfying the threshold.

In this manner, the medical device system may facilitate the medical intervention by a clinician who may take action, such as titrating or changing a medication of the patient. In some examples, a medical device system includes: a plurality of electrodes; sensing circuitry configured to sense an ECG of a patient; and processing circuitry configured to: determine a T-wave morphology in the ECG; based on the T-wave morphology, determine an estimate of serum potassium in blood of the patient; determine that the estimate of serum potassium in the blood satisfies a threshold; and based on the estimate of serum potassium in the blood satisfying the threshold, generate an indication for output that is based at least in part on the estimate of serum potassium in the blood satisfying the threshold.

In some examples, a method includes determining a T-wave morphology in an ECG of a patient; based on the T-wave morphology, determining an estimate of serum potassium in blood of the patient; determining that the estimate of serum potassium in the blood satisfies a threshold; and based on the estimate of serum potassium in the blood satisfying the threshold, generating an indication for output that is based at least in part on the estimate of serum potassium in the blood satisfying the threshold.

In some examples, a non-transitory computer-readable medium includes instructions for causing one or more processors to: determine a T-wave morphology in an ECG of a patient; based on the T-wave morphology, determine an estimate of serum potassium in blood of the patient; determine that the estimate of serum potassium in the blood satisfies a threshold; and based on the estimate of serum potassium in the blood satisfying the threshold, generate an indication for output that is based at least in part on the estimate of serum potassium in the blood satisfying the threshold.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 illustrates the environment of an example medical device system in conjunction with a patient, in accordance with one or more techniques of this disclosure.

FIG. 2 is a conceptual drawing illustrating an example configuration of the implantable medical device (IMD) of the medical device system of FIG. 1, in accordance with one or more techniques described herein.

FIG. 3 is a functional block diagram illustrating an example configuration of the IMD of FIGS. 1 and 2, in accordance with one or more techniques described herein.

FIGS. 4A and 4B are block diagrams illustrating two additional example IMDs that may be substantially similar to the IMD of FIGS. 1-3, but which may include one or more additional features, in accordance with one or more techniques described herein.

FIG. 5 is a block diagram illustrating an example configuration of components of the external device of FIG. 1, in accordance with one or more techniques of this disclosure.

FIG. 6 is a block diagram illustrating an example system that includes an access point, a network, external computing devices, such as a server, and one or more other computing devices, which may be coupled to the IMD of FIGS. 1-4, an external device, and processing circuitry via a network, in accordance with one or more techniques described herein.

FIG. 7 is a conceptual diagram of an example portion of an ECG.

FIG. 8 is a conceptual diagram of example ECGs taken pre-dialysis and post-dialysis.

FIG. 9 is a conceptual drawing of example morphological features of an ECG which may be used to determine an estimate of serum potassium in accordance with one or more techniques described herein.

FIG. 10 is a graphical diagram of example data for a patient-specific machine learned model in accordance with one or more techniques described herein.

FIG. 11 is a graphical diagram of an example average error for a patient specific machine learned model per the size of a training set in accordance with one or more techniques described herein.

FIG. 12 is a graphical diagram of example data for a population-averaged machine learned model in accordance with one or more techniques described herein.

FIG. 13 is a graphical diagram of an example average error for a population-averaged machine learned model per the size of a training set in accordance with one or more techniques described herein.

FIG. 14 is a graphical diagram of Poincaré plots in accordance with one or more techniques described herein.

FIG. 15 is a graphical diagram of Lorenz plots in accordance with one or more techniques described herein.

FIG. 16 is a graphical diagram illustrating examples of estimation of GFR based on HRV in an ECG in accordance with one or more techniques described herein.

FIG. 17 is a tabular diagram illustrating an example of estimated GFR and serum creatine in accordance with one or more techniques described herein.

FIG. 18 is a flow diagram illustrating an example of dynamically adjusting an impedance measurement range in accordance with one or more techniques of this disclosure.

Like reference characters denote like elements throughout the description and figures.

### DETAILED DESCRIPTION

This disclosure describes techniques for estimating a serum potassium in blood of a patient and/or kidney function of a patient. Serum potassium, kidney function, and blood pressure are physiological parameters that cardiologists may use to manage or titrate heart failure medication, such as angiotensin-converting enzyme (ACE)-inhibitors and/or angiotensin II receptor blockers (ARBs) for hyperkalemia, diuretics for hypokalemia, beta-blockers, or the like. The techniques of this disclosure may facilitate the monitoring and management of a heart failure patient, chronic kidney disease patient, or other patient by a clinician in a manner that may be non-invasive, remote, and/or continuous. By providing for the non-invasive, remote, and/or continuous monitoring of serum potassium and/or kidney function, the techniques of this disclosure may facilitate early intervention by a clinician during deteriorating conditions of the patient, faster detection of actionable events, reduced hospitalization, better management of heart failure medications.

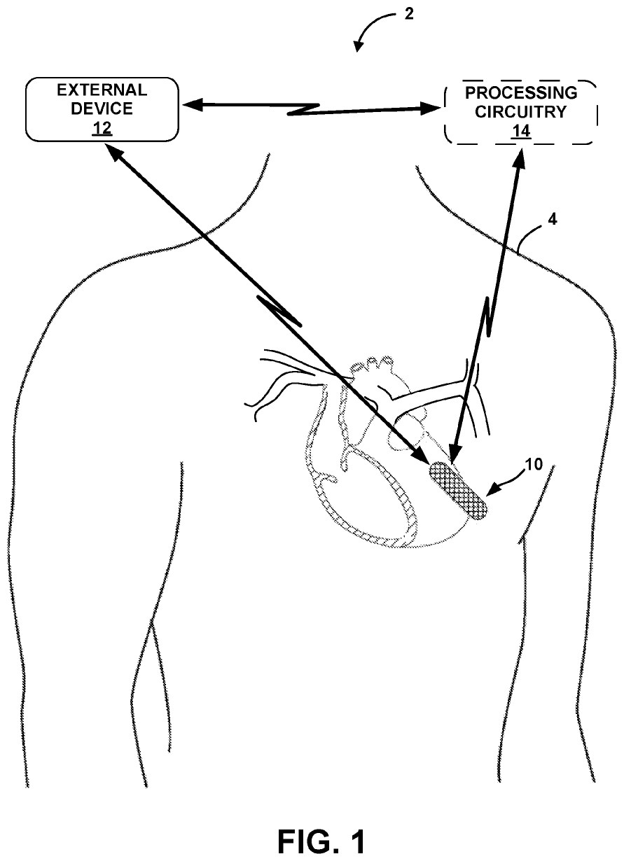

FIG. 1 illustrates the environment of an example medical device system 2 in conjunction with a patient 4, in accordance with one or more techniques of this disclosure. While the techniques described herein are generally described in the context of an insertable cardiac monitor, the techniques of this disclosure may be implemented in any implantable medical device configured to sense an ECG of a patient, such as a cardiac pacemaker, defibrillator, a ventricular assist device, or the like. The example techniques may be used with an IMD 10, which may be in wireless communication with at least one of external device 12 and other devices not pictured in FIG. 1. Processing circuitry 14 is conceptually illustrated in FIG. 1 as separate from IMD 10 and external device 12, but may be processing circuitry of IMD 10 and/or processing circuitry of external device 12. In general, the techniques of this disclosure may be performed by processing circuitry 14 of one or more devices of a system, such as one or more devices that include sensors that provide signals, or processing circuitry of one or more devices that do not include sensors, but nevertheless analyze signals using the techniques described herein. For example, another external device (not pictured in FIG. 1) may include at least a portion of processing circuitry 14, the other external device configured for remote communication with IMD 10 and/or external device 12 via a network.

In some examples, IMD 10 is implanted outside of a thoracic cavity of patient 4 (e.g., subcutaneously in the pectoral location illustrated in FIG. 1). IMD 10 may be positioned near the sternum near or just below the level of patient 4's heart, e.g., at least partially within the cardiac silhouette. In some examples, IMD 10 takes the form of a LINQ™ Insertable Cardiac Monitor (ICM), available from Medtronic plc, of Dublin, Ireland.

Clinicians sometimes diagnose patients with medical conditions based on one or more observed physiological signals collected by physiological sensors, such as electrodes, optical sensors, chemical sensors, temperature sensors, acoustic sensors, and motion sensors. In some cases, clinicians apply non-invasive sensors to patients in order to sense one or more physiological signals while a patient is in a clinic for a medical appointment. However, in some examples, physiological markers (e.g., arrythmia, etc.) of a patient condition are rare or are difficult to observe over a relatively short period of time. As such, in these examples, a clinician may be unable to observe the physiological markers needed to diagnose a patient with a medical condition or effectively treat the patient while monitoring one or more physiological signals of the patient during a medical appointment. In the example illustrated in FIG. 1, IMD 10 is implanted within patient 4 to continuously record one or more physiological signals, such as an ECG, of patient 4 over an extended period of time.

In some examples, IMD 10 includes a plurality of electrodes. The plurality of electrodes is configured to detect signals that enable processing circuitry 14, e.g., of IMD 10, to monitor and/or record physiological parameters of patient 4. For example, the plurality of electrodes may be configured to sense an ECG of patient 4. IMD 10 may additionally or alternatively include one or more optical sensors, accelerometers, temperature sensors, chemical sensors, light sensors, pressure sensors, in some examples. Such sensors may detect one or more physiological parameters indicative of a patient condition.

According to the techniques of this disclosure, IMD 10, external device 12, and/or processing circuitry 14 may use the sensed ECG to determine an estimate of a serum potassium in blood of patient 4. For example, IMD 10, external device 12, and/or processing circuitry 14 may determine a T-wave morphology in the ECG and based on the T-wave morphology, determine an estimate of the serum potassium in the blood of patient. IMD 10, external device 12, and/or processing circuitry 14 may determine that the estimate of serum potassium in the blood satisfies a threshold, and based on the estimate of serum potassium in the blood satisfying the threshold, generate an indication for output that is based at least in part on the estimate of serum potassium in the blood satisfying the threshold. For example, the estimate of serum potassium may satisfy the exacerbation threshold by being greater than, greater than or equal to, equal to, less than, or less than or equal to the threshold. The indication may include an alert, the estimate of the serum potassium, a recommendation for remedial action (e.g., a recommendation to alter the medication or dosage of medication of patient 4), or the like.

In some examples, based on the estimate of serum potassium in the blood satisfying the threshold, IMD 10, external device 12, and/or processing circuitry 14 may control the sensing circuitry to increase a sampling rate of the ECG and monitor the ECG for arrythmia of a heart of the patient. In some examples, IMD 10, external device 12, and/or processing circuitry 14 may determine an arrythmia of the heart of patient 4 and based on determining the arrythmia of the heart of patient 4, perform at least one of pace the heart of patient 4 or generate an indication of arrythmia for output.

In some examples, IMD 10, external device 12, and/or processing circuitry 14 may determine an R-wave morphology in the ECG, the R-wave preceding the T-wave and normalize the T-wave morphology based on the R-wave morphology prior to determining the estimate of serum potassium in the blood. In some examples, IMD 10, external device 12, and/or processing circuitry 14 may average the normalized T-wave across several successive heart beats, such as over a 30 second period or longer.

In some examples, IMD 10, external device 12, and/or processing circuitry 14 may apply at least one of a patient specific machine learned model or a population averaged machine learned model to the morphology of the T-wave when determining the estimate of the serum potassium in the blood. For example, a patient specific machine learned model may be trained using data collected from a single patient (e.g., patient 4). In the case of a patient specific machine learned model, the model may be applicable only to the patient whose data was used to train the model. A population averaged machine learned model, on the other hand, may be trained using data collected from a plurality of patients. Such a model may be applicable to a plurality of patients.

According to the techniques of this disclosure, IMD 10, external device 12, and/or processing circuitry 14 may use the sensed ECG to determine an estimate of GFR of patient 4. Kidney function is known to be related to autonomic nervous activity. Some aspects of autonomic nervous activity can be observed in heart rate variability (HRV). For example, IMD 10, external device 12, and/or processing circuitry 14 may monitor a plurality of quantitative metrics, and in some examples, at least one qualitative metric, to capture autonomic nervous activity in HRV of patient 4 and use such metrics to estimate GFR. In some examples, linear regression or machine learning techniques may be employed to improve the accuracy of the estimates of GFR. In some examples, time of day, activity level, heart rate, and or temperature may also be used to determine the estimate of GFR.

For example, IMD 10, external device 12, and/or processing circuitry 14 may utilize Poincaré plots as a geometric, nonlinear technique to assess dynamics of HRV and to estimate GFR. A Poincaré plot is a recurrence plot that may be used to quantify self-similarity in processes. Additionally, or alternatively, IMD 10, external device 12, and/or processing circuitry 14 may use Lorenz plots to estimate GFR. Lorenz plots are scatterplots that show an R-R interval as a function of preceding R-R intervals. Lorenz plots are similar to Poincaré plots, but provide an orthogonal perspective on variability in collected data. IMD 10, external device 12, and/or processing circuitry 14 may use linear regression or machine learning techniques with the collected data to determine the estimate of GFR. For example, the linear regression techniques may be univariable and/or multivariable techniques.

External device 12 may be a hand-held computing device with a display viewable by the user and an interface for providing input to external device 12 (i.e., a user input mechanism). For example, external device 12 may include a small display screen (e.g., a liquid crystal display (LCD) or a light emitting diode (LED) display) that presents information to the user. In addition, external device 12 may include a touch screen display, keypad, buttons, a peripheral pointing device, voice activation, or another input mechanism that allows the user to navigate through the user interface of external device 12 and provide input. If external device 12 includes buttons and a keypad, the buttons may be dedicated to performing a certain function, e.g., a power button, the buttons and the keypad may be soft keys that change in function depending upon the section of the user interface currently viewed by the user, or any combination thereof.

In other examples, external device 12 may be a larger workstation or a separate application within another multi-function device, rather than a dedicated computing device. For example, the multi-function device may be a notebook computer, tablet computer, workstation, one or more servers, cellular phone, personal digital assistant, or another computing device that may run an application that enables the computing device to operate as a secure device.

When external device 12 is configured for use by the clinician, external device 12 may be used to transmit instructions to IMD 10 and to receive measurements, such as an ECG of patient 4, an estimate of serum potassium, a measure of heart rate variability, or an estimate of GFR. Example instructions may include requests to set electrode combinations for sensing and any other information that may be useful for programming into IMD 10. The clinician may also configure and store operational parameters for IMD 10 within IMD 10 with the aid of external device 12. In some examples, external device 12 assists the clinician in the configuration of IMD 10 by providing a system for identifying potentially beneficial operational parameter values.

Whether external device 12 is configured for clinician or patient use, external device 12 is configured to communicate with IMD 10 and, optionally, another computing device (not illustrated in FIG. 1), via wireless communication. External device 12, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth® specification sets, or other communication technologies operable at ranges greater than near-field communication technologies).

Processing circuitry 14, in some examples, may include one or more processors that are configured to implement functionality and/or process instructions for execution within IMD 10. For example, processing circuitry 14 may be capable of processing instructions stored in a storage device. Processing circuitry 14 may include, for example, microprocessors, digital signal processors (DSPs), application specific integrated circuits (ASICs), field-programmable gate arrays (FPGAs), or equivalent discrete or integrated logic circuitry, or a combination of any of the foregoing devices or circuitry. Accordingly, processing circuitry 14 may include any suitable structure, whether in hardware, software, firmware, or any combination thereof, to perform the functions ascribed herein to processing circuitry 14.

Processing circuitry 14 may represent processing circuitry located within any combination of IMD 10 and external device 12. In some examples, processing circuitry 14 may be entirely located within a housing of IMD 10. In other examples, processing circuitry 14 may be entirely located within a housing of external device 12. In other examples, processing circuitry 14 may be located within any combination of IMD 10, external device 12, and another device or group of devices that are not illustrated in FIG. 1. As such, techniques and capabilities attributed herein to processing circuitry 14 may be attributed to any combination of IMD 10, external device 12, and other devices that are not illustrated in FIG. 1.

In some examples, IMD 10 includes one or more accelerometers. An accelerometer of IMD 10 may collect an accelerometer signal which reflects a measurement of a motion of patient 4. In some cases, the accelerometer may collect a three-axis accelerometer signal indicative of patient 4's movements within a three-dimensional Cartesian space. For example, the accelerometer signal may include a vertical axis accelerometer signal vector, a lateral axis accelerometer signal vector, and a frontal axis accelerometer signal vector. The vertical axis accelerometer signal vector may represent an acceleration of patient 4 along a vertical axis, the lateral axis accelerometer signal vector may represent an acceleration of patient 4 along a lateral axis, and the frontal axis accelerometer signal vector may represent an acceleration of patient 4 along a frontal axis. In some cases, the vertical axis substantially extends along a torso of patient 4 when patient 4 from a neck of patient 4 to a waist of patient 4, the lateral axis extends across a chest of patient 4 perpendicular to the vertical axis, and the frontal axis extends outward from and through the chest of patient 4, the frontal axis being perpendicular to the vertical axis and the lateral axis. In some examples, processing circuitry 14 may be configured to identify, based on one of more accelerometer signals, a posture of patient 4. In some examples, the estimate of the serum potassium may be based in part on the posture of patient 4. In some examples, the posture of patient 4 may be determined by processing circuity 14 as prone, supine, upright, lateral recumbent, Fowler's, or other posture.

Although in one example IMD 10 takes the form of an ICM, in other examples, IMD 10 takes the form of any combination of implantable cardioverter defibrillators (ICDs) with intravascular or extravascular leads, pacemakers, cardiac resynchronization therapy devices (CRT-Ds), ventricular assist devices (VADs), or neurostimulators, as examples. The ECG of the patient, the estimate of the serum potassium, the HRV, and/or the estimate of GFR may be sensed or determined using one or more of the aforementioned devices.

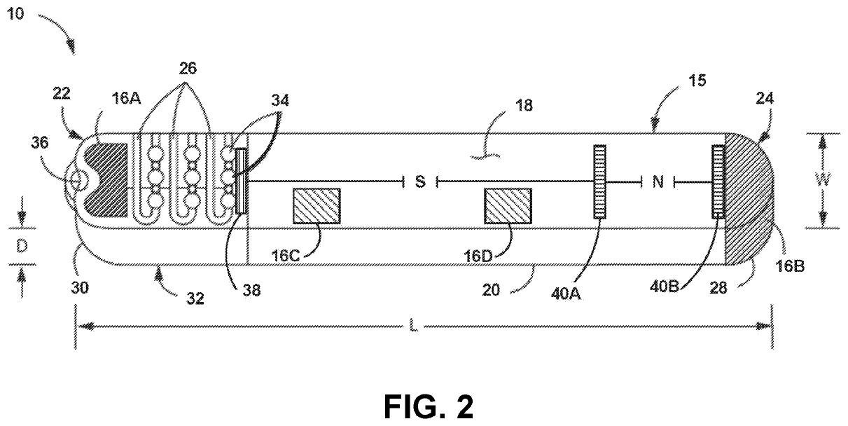

FIG. 2 is a conceptual drawing illustrating an example configuration of IMD 10 of the medical device system 2 of FIG. 1, in accordance with one or more techniques described herein. In the example shown in FIG. 2, IMD 10 may include a leadless, subcutaneously-implantable monitoring device having housing 15, proximal electrode 16A, and distal electrode 16B. Housing 15 may further include first major surface 18, second major surface 20, proximal end 22, and distal end 24. In some examples, IMD 10 may include one or more additional electrodes 16C, 16D positioned on one or both of major surfaces 18, 20 of IMD 10. Housing 15 encloses electronic circuitry located inside the IMD 10, and protects the circuitry contained therein from fluids such as body fluids. In some examples, electrical feedthroughs provide electrical connection of electrodes 16A-16D, and antenna 26, to circuitry within housing 15. In some examples, electrode 16B may be formed from an uninsulated portion of conductive housing 15.

In the example shown in FIG. 2, IMD 10 is defined by a length L, a width W, and thickness or depth D. In this example, IMD 10 is in the form of an elongated rectangular prism in which length L is significantly greater than width W, and in which width W is greater than depth D. However, other configurations of IMD 10 are contemplated, such as those in which the relative proportions of length L, width W, and depth D vary from those described and shown in FIG. 2. In some examples, the geometry of the IMD 10, such as the width W being greater than the depth D, may be selected to allow IMD 10 to be inserted under the skin of the patient using a minimally invasive procedure and to remain in the desired orientation during insertion. In addition, IMD 10 may include radial asymmetries (e.g., the rectangular shape) along a longitudinal axis of IMD 10, which may help maintain the device in a desired orientation following implantation.

In some examples, a spacing between proximal electrode 16A and distal electrode 16B may range from about 30-55 mm, about 35-55 mm, or about 40-55 mm, or more generally from about 25-60 mm. Overall, IMD 10 may have a length L of about 20-30 mm, about 40-60 mm, or about 45-60 mm. In some examples, the width W of major surface 18 may range from about 3-10 mm, and may be any single width or range of widths between about 3-10 mm. In some examples, a depth D of IMD 10 may range from about 2-9 mm. In other examples, the depth D of IMD 10 may range from about 2-5 mm, and may be any single or range of depths from about 2-9 mm. In any such examples, IMD 10 is sufficiently compact to be implanted within the subcutaneous space of patient 4 in the region of a pectoral muscle.

IMD 10, according to an example of the present disclosure, may have a geometry and size designed for ease of implant and patient comfort. Examples of IMD 10 described in this disclosure may have a volume of 3 cubic centimeters (cm³) or less, 1.5 cm³ or less, or any volume therebetween. In addition, in the example shown in FIG. 2, proximal end 22 and distal end 24 are rounded to reduce discomfort and irritation to surrounding tissue once implanted under the skin of patient 4.

In the example shown in FIG. 2, first major surface 18 of IMD 10 faces outward towards the skin, when IMD 10 is inserted within patient 4, whereas second major surface 20 is faces inward toward musculature of patient 4. Thus, first and second major surfaces 18, 20 may face in directions along a sagittal axis of patient 4 (see FIG. 1), and this orientation may be generally maintained upon implantation due to the dimensions of IMD 10.

Proximal electrode 16A and distal electrode 16B may be used to sense cardiac EGM signals (e.g., ECG signals) when IMD 10 is implanted subcutaneously in patient 4. In some examples, processing circuitry of IMD 10 also may determine whether cardiac ECG signals of patient 4 are indicative of arrhythmia or other abnormalities, which processing circuitry of IMD 10 may evaluate in determining whether a medical condition (e.g., heart failure, sleep apnea, or COPD) of patient 4 has changed. The cardiac ECG signals may be stored in a memory of IMD 10, and data derived from the cardiac ECG signals, such as estimates of serum potassium, HRV and/or the estimates of GFR may be transmitted via integrated antenna 26 to another device, such as external device 12. Additionally, in some examples, electrodes 16A, 16B may be used by communication circuitry of IMD 10 for tissue conductance communication (TCC) communication with external device 12 or another device.

In the example shown in FIG. 2, proximal electrode 16A is in close proximity to proximal end 22, and distal electrode 16B is in close proximity to distal end 24 of IMD 10. In this example, distal electrode 16B is not limited to a flattened, outward facing surface, but may extend from first major surface 18, around rounded edges 28 or end surface 30, and onto the second major surface 20 in a three-dimensional curved configuration. As illustrated, proximal electrode 16A is located on first major surface 18 and is substantially flat and outward facing. However, in other examples not shown here, proximal electrode 16A and distal electrode 16B both may be configured like proximal electrode 16A shown in FIG. 2, or both may be configured like distal electrode 16B shown in FIG. 2. In some examples, additional electrodes 16C and 16D may be positioned on one or both of first major surface 18 and second major surface 20, such that a total of four electrodes are included on IMD 10. Any of electrodes 16A-16D may be formed of a biocompatible conductive material. For example, any of electrodes 16A-16D may be formed from any of stainless steel, titanium, platinum, iridium, or alloys thereof. In addition, electrodes of IMD 10 may be coated with a material such as titanium nitride or fractal titanium nitride, although other suitable materials and coatings for such electrodes may be used.

In the example shown in FIG. 2, proximal end 22 of IMD 10 includes header assembly 32 having one or more of proximal electrode 16A, integrated antenna 26, anti-migration projections 34, and suture hole 36. Integrated antenna 26 is located on the same major surface (e.g., first major surface 18) as proximal electrode 16A, and may be an integral part of header assembly 32. In other examples, integrated antenna 26 may be formed on the major surface opposite from proximal electrode 16A, or, in still other examples, may be incorporated within housing 15 of IMD 10. Antenna 26 may be configured to transmit or receive electromagnetic signals for communication. For example, antenna 26 may be configured to transmit to or receive signals from a programmer via inductive coupling, electromagnetic coupling, tissue conductance, Near Field Communication (NFC), Radio Frequency Identification (RFID), Bluetooth®, WiFi®, or other proprietary or non-proprietary wireless telemetry communication schemes. Antenna 26 may be coupled to communication circuitry of IMD 10, which may drive antenna 26 to transmit signals to external device 12, and may transmit signals received from external device 12 to processing circuitry of IMD 10 via communication circuitry.

In some examples, IMD 10 may include several features for retaining IMD 10 in position once subcutaneously implanted in patient 4, so as to decrease the chance that IMD 10 migrates in the body of patient 4. For example, as shown in FIG. 2, housing 15 may include anti-migration projections 34 positioned adjacent integrated antenna 26. Anti-migration projections 34 may include a plurality of bumps or protrusions extending away from first major surface 18, and may help prevent longitudinal movement of IMD 10 after implantation in patient 4. In other examples, anti-migration projections 34 may be located on the opposite major surface as proximal electrode 16A and/or integrated antenna 26. In addition, in the example shown in FIG. 2 header assembly 32 includes suture hole 36, which provides another means of securing IMD 10 to the patient to prevent movement following insertion. In the example shown, suture hole 36 is located adjacent to proximal electrode 16A. In some examples, header assembly 32 may include a molded header assembly made from a polymeric or plastic material, which may be integrated or separable from the main portion of IMD 10.

In the example shown in FIG. 2, IMD 10 includes a light emitter 38, a proximal light detector 40A, and a distal light detector 40B positioned on housing 15 of IMD 10. Light detector 40A may be positioned at a distance S from light emitter 38, and a distal light detector 40B positioned at a distance S+N from light emitter 38. In other examples, IMD 10 may include only one of light detectors 40A, 40B, or may include additional light emitters and/or additional light detectors. Although light emitter 38 and light detectors 40A, 40B are described herein as being positioned on housing 15 of IMD 10, in other examples, one or more of light emitter 38 and light detectors 40A, 40B may be positioned, on a housing of another type of IMD within patient 4, such as a transvenous, subcutaneous, or extravascular pacemaker or ICD, or connected to such a device via a lead.

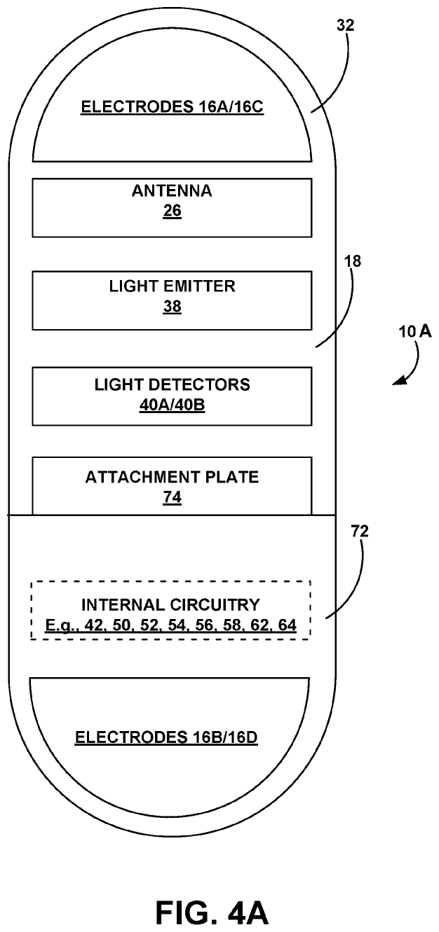

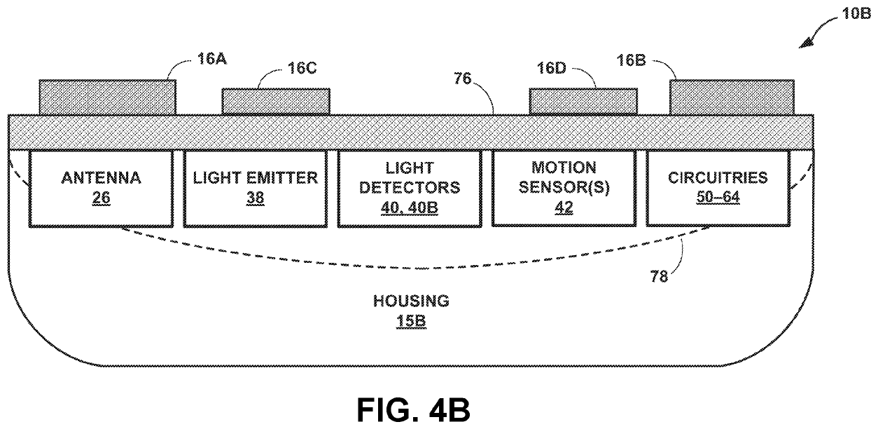

As shown in FIG. 2, light emitter 38 may be positioned on header assembly 32, although, in other examples, one or both of light detectors 40A, 40B may additionally or alternatively be positioned on header assembly 32. In some examples, light emitter 38 may be positioned on a medial section of IMD 10, such as part way between proximal end 22 and distal end 24. Although light emitter 38 and light detectors 40A, 40B are illustrated as being positioned on first major surface 18, light emitter 38 and light detectors 40A, 40B alternatively may be positioned on second major surface 20. In some examples, IMD may be implanted such that light emitter 38 and light detectors 40A, 40B face inward when IMD 10 is implanted, toward the muscle of patient 4, which may help minimize interference from background light coming from outside the body of patient 4. Light detectors 40A, 40B may include a glass or sapphire window, such as described below with respect to FIG. 4B, or may be positioned beneath a portion of housing 15 of IMD 10 that is made of glass or sapphire, or otherwise transparent or translucent.

In some examples, IMD 10 may include one or more additional sensors, such as one or more accelerometers (not shown in FIG. 2). Such accelerometers may be 3D accelerometers configured to generate signals indicative of one or more types of movement of the patient, such as gross body movement (e.g., motion) of the patient, patient posture, movements associated with the beating of the heart, or coughing, rales, or other respiration abnormalities, or the movement of IMD 10 within the body of patient 4. One or more of the parameters monitored by IMD 10 (e.g., bio impedance, EGM) may fluctuate in response to changes in one or more such types of movement. For example, changes in parameter values sometimes may be attributable to increased patient motion (e.g., exercise or other physical motion as compared to immobility) or to changes in patient posture, and not necessarily to changes in a medical condition. Thus, in some methods of identifying or tracking a medical condition of patient 4, it may be advantageous to account for such fluctuations when determining whether a change in a parameter is indicative of a change in a medical condition.

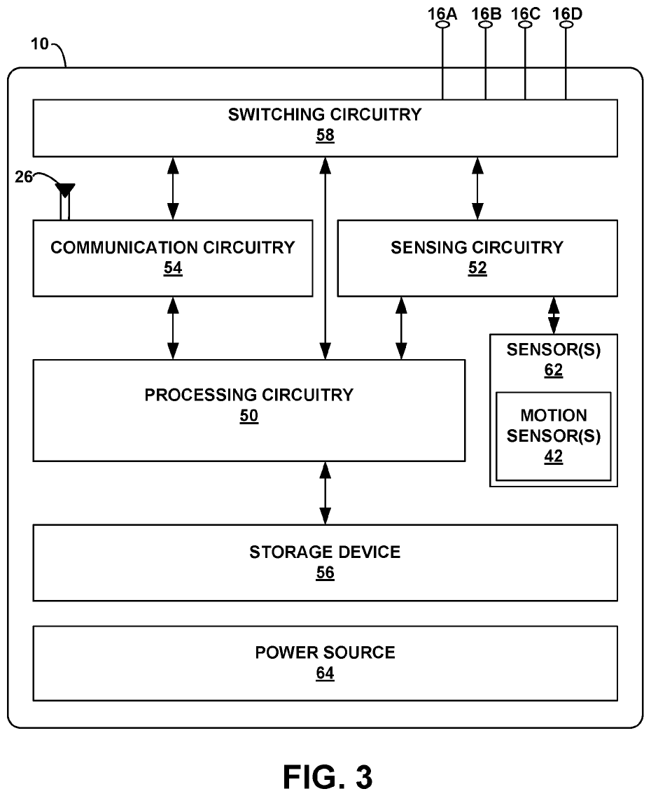

FIG. 3 is a functional block diagram illustrating an example configuration of IMD 10 of FIGS. 1 and 2, in accordance with one or more techniques described herein. In the illustrated example, IMD 10 includes electrodes 16, antenna 26, processing circuitry 50, sensing circuitry 52, communication circuitry 54, storage device 56, switching circuitry 58, sensors 62 including motion sensor(s) 42 (which may be an accelerometer), and power source 64. Although not illustrated in FIG. 3, sensors 62 may include light detectors 40 of FIG. 2.

Processing circuitry 50 may include fixed function circuitry and/or programmable processing circuitry. Processing circuitry 50 may include any one or more of a microprocessor, a controller, a DSP, an ASIC, an FPGA, or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof. In some examples, one or more techniques of this disclosure may be performed by processing circuitry 50.

Sensing circuitry 52 and communication circuitry 54 may be selectively coupled to electrodes 16A-16D via switching circuitry 58, as controlled by processing circuitry 50. Sensing circuitry 52 may monitor signals from electrodes 16A-16D in order to monitor electrical activity of heart (e.g., to produce an ECG). Sensing circuitry 52 also may monitor signals from sensors 62, which may include motion sensor(s) 42 (which may be an accelerometer), and any additional light detectors that may be positioned on IMD 10. In some examples, sensing circuitry 52 may include one or more filters and amplifiers for filtering and amplifying signals received from one or more of electrodes 16A-16D and/or motion sensor(s) 42 (which may be an accelerometer).

Communication circuitry 54 may include any suitable hardware, firmware, software or any combination thereof for communicating with another device, such as external device 12 or another IMD or sensor, such as a pressure sensing device. Under the control of processing circuitry 50, communication circuitry 54 may receive downlink telemetry from, as well as send uplink telemetry to, external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network developed by Medtronic, plc, of Dublin, Ireland.

A clinician or other user may retrieve data from IMD 10 using external device 12, or by using another local or networked computing device configured to communicate with processing circuitry 50 via communication circuitry 54. The clinician may also program parameters of IMD 10 using external device 12 or another local or networked computing device.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Power source 64 is configured to deliver operating power to the components of IMD 10. Power source 64 may include a battery and a power generation circuit to produce the operating power. In some examples, the battery is rechargeable to allow extended operation. In some examples, recharging is accomplished through proximal inductive interaction between an external charger and an inductive charging coil within external device 12. Power source 64 may include any one or more of a plurality of different battery types, such as nickel cadmium batteries and lithium ion batteries. A non-rechargeable battery may be selected to last for several years, while a rechargeable battery may be inductively charged from an external device, e.g., on a daily or weekly basis.

FIGS. 4A and 4B illustrate two additional example IMDs that may be substantially similar to IMD 10 of FIGS. 1-3, but which may include one or more additional features, in accordance with one or more techniques described herein. The components of FIGS. 4A and 4B may not necessarily be drawn to scale, but instead may be enlarged to show detail. FIG. 4A is a block diagram of a top view of an example configuration of an IMD 10A. FIG. 4B is a block diagram of a side view of example IMD 10B, which may include an insulative layer as described below.

FIG. 4A is a conceptual drawing illustrating another example IMD 10A that may be substantially similar to IMD 10 of FIG. 1. In addition to the components illustrated in FIGS. 1-3, the example of IMD 10 illustrated in FIG. 4A also may include a body portion 72 and an attachment plate 74. Attachment plate 74 may be configured to mechanically couple header assembly 32 to body portion 72 of IMD 10A. Body portion 72 of IMD 10A may be configured to house one or more of the internal components of IMD 10 illustrated in FIG. 3, such as one or more of processing circuitry 50, sensing circuitry 52, communication circuitry 54, storage device 56, switching circuitry 58, internal components of sensors 62, and power source 64. In some examples, body portion 72 may be formed of one or more of titanium, ceramic, or any other suitable biocompatible materials.

FIG. 4B is a conceptual drawing illustrating another example IMD 10B that may include components substantially similar to IMD 10 of FIG. 1. In addition to the components illustrated in FIGS. 1-3, the example of IMD 10B illustrated in FIG. 4B also may include a wafer-scale insulative cover 76, which may help insulate electrical signals passing between electrodes 16A-16D and/or light detectors 40A, 40B on housing 15B and processing circuitry 50. In some examples, insulative cover 76 may be positioned over an open housing 15 to form the housing for the components of IMD 10B. One or more components of IMD 10B (e.g., antenna 26, light emitter 38, light detectors 40A, 40B, processing circuitry 50, sensing circuitry 52, communication circuitry 54, switching circuitry 58, and/or power source 64) may be formed on a bottom side of insulative cover 76, such as by using flip-chip technology. Insulative cover 76 may be flipped onto a housing 15B. When flipped and placed onto housing 15B, the components of IMD 10B formed on the bottom side of insulative cover 76 may be positioned in a gap 78 defined by housing 15B.

Insulative cover 76 may be configured so as not to interfere with the operation of IMD 10B. For example, one or more of electrodes 16A-16D may be formed or placed above or on top of insulative cover 76, and electrically connected to switching circuitry 58 through one or more vias (not shown) formed through insulative cover 76. Insulative cover 76 may be formed of sapphire (i.e., corundum), glass, parylene, and/or any other suitable insulating material. Sapphire may be greater than 80% transmissive for wavelengths in the range of about 300 nm to about 4000 nm, and may have a relatively flat profile. In the case of variation, different transmissions at different wavelengths may be compensated for, such as by using a ratiometric approach. In some examples, insulative cover 76 may have a thickness of about 300 micrometers to about 600 micrometers. Housing 15B may be formed from titanium or any other suitable material (e.g., a biocompatible material), and may have a thickness of about 200 micrometers to about 500 micrometers. These materials and dimensions are examples only, and other materials and other thicknesses are possible for devices of this disclosure.

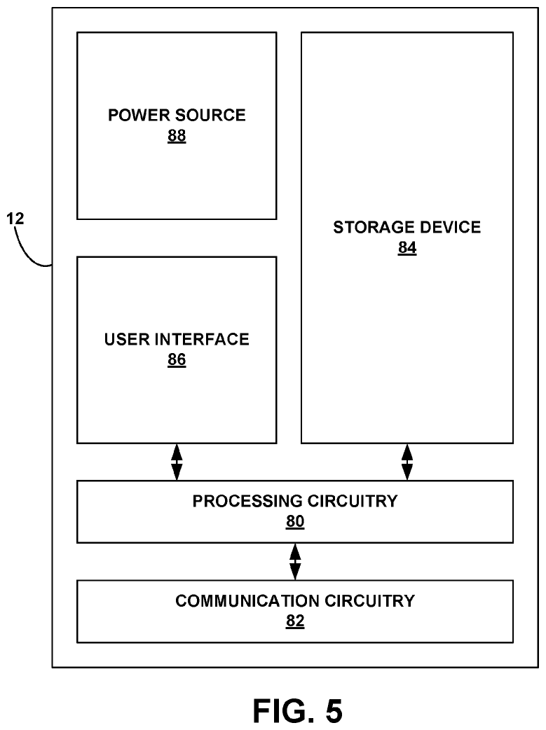

FIG. 5 is a block diagram illustrating an example configuration of components of external device 12, in accordance with one or more techniques of this disclosure. In the example of FIG. 5, external device 12 includes processing circuitry 80, communication circuitry 82, storage device 84, user interface 86, and power source 88.

Processing circuitry 80, in one example, may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80. In some examples, processing circuitry 80 may perform one or more of the techniques of this disclosure.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, dynamic random access memories (DRAM), static random access memories (SRAM), magnetic discs, optical discs, flash memories, or forms of electrically programmable memories (EPROM) or EEPROM. In some examples, storage device 84 is used to store data indicative of instructions for execution by processing circuitry 80. Storage device 84 may be used by software or applications running on external device 12 to temporarily store information during program execution.

Data exchanged between external device 12 and IMD 10 may include operational parameters. External device 12 may transmit data including computer readable instructions which, when implemented by IMD 10, may control IMD 10 to change one or more operational parameters and/or export collected data. For example, processing circuitry 80 may transmit an instruction to IMD 10 which requests IMD 10 to export collected data (e.g., data corresponding to one or more of an ECG signal or portion thereof, an estimate of serum potassium in the blood of patient 4, HRV of patient 4, an estimate of GFR of patient 4, an accelerometer signal, or other collected data) to external device 12. In turn, external device 12 may receive the collected data from IMD 10 and store the collected data in storage device 84. Additionally, or alternatively, processing circuitry 80 may export instructions to IMD 10 requesting IMD 10 to update one or more operational parameters of IMD 10.

A user, such as a clinician or patient 4, may interact with external device 12 through user interface 86. User interface 86 includes a display (not shown), such as an LCD or LED display or other type of screen, with which processing circuitry 80 may present information related to IMD 10 (e.g., EGM or ECG signals obtained from at least one electrode or at least one electrode combination, serum potassium values, estimated GFR, etc.). In addition, user interface 86 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to patient 4, receiving voice commands from patient 4, or both. Storage device 84 may include instructions for operating user interface 86 and for managing power source 88.

Power source 88 is configured to deliver operating power to the components of external device 12. Power source 88 may include a battery and a power generation circuit to produce the operating power. In some examples, the battery is rechargeable to allow extended operation. Recharging may be accomplished by electrically coupling power source 88 to a cradle or plug that is connected to an alternating current (AC) outlet. In addition, recharging may be accomplished through proximal inductive interaction between an external charger and an inductive charging coil within external device 12. In other examples, traditional batteries (e.g., nickel cadmium or lithium ion batteries) may be used. In addition, external device 12 may be directly coupled to an alternating current outlet to operate.

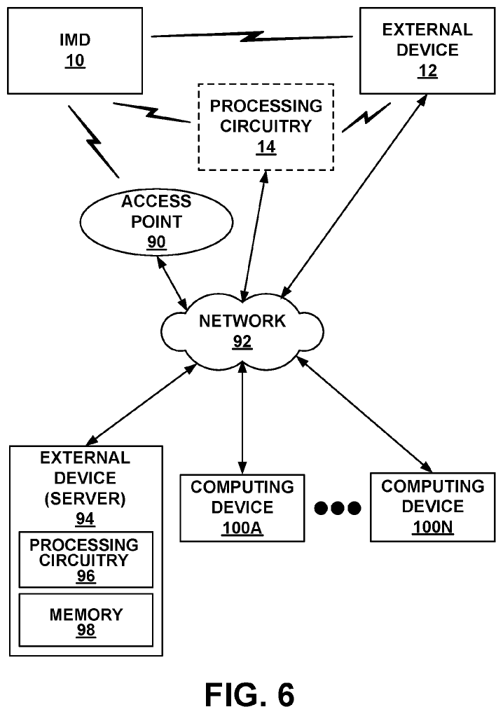

FIG. 6 is a block diagram illustrating an example system that includes an access point 90, a network 92, external computing devices, such as a server 94, and one or more other computing devices 100A-100N, which may be coupled to IMD 10, external device 12, and processing circuitry 14 via network 92, in accordance with one or more techniques described herein. In this example, IMD 10 may use communication circuitry 54 to communicate with external device 12 via a first wireless connection, and to communication with an access point 90 via a second wireless connection. In the example of FIG. 6, access point 90, external device 12, server 94, and computing devices 100A-100N are interconnected and may communicate with each other through network 92.

Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with the patient. As discussed above, IMD 10 may be configured to transmit data, such as any one or combination of an ECG signal or portion thereof, an estimate of serum potassium in the blood of patient 4, HRV of patient 4, an estimate of GFR of patient 4, an accelerometer signal, or other data collected by IMD 10 to external device 12. In addition, access point 90 may interrogate IMD 10, such as periodically or in response to a command from the patient or network 92, in order to retrieve parameter values determined by processing circuitry 50 of IMD 10, or other operational or patient data from IMD 10. Access point 90 may then communicate the retrieved data to server 94 via network 92.

In some cases, server 94 may be configured to provide a secure storage site for data that has been collected from IMD 10, and/or external device 12, such as ECGs, estimates of serum potassium, HRVs, and/or estimates of GFR. In some cases, server 94 may assemble data in web pages or other documents for viewing by trained professionals, such as clinicians, via computing devices 100A-100N. One or more aspects of the illustrated system of FIG. 6 may be implemented with general network technology and functionality, which may be similar to that provided by the Medtronic CareLink® Network developed by Medtronic plc, of Dublin, Ireland.

Server 94 may include processing circuitry 96. Processing circuitry 96 may include fixed function circuitry and/or programmable processing circuitry. Processing circuitry 96 may include any one or more of a microprocessor, a controller, a DSP, an ASIC, an FPGA, or equivalent discrete or analog logic circuitry. In some examples, processing circuitry 96 may include multiple components, such as any combination of one or more microprocessors, one or more controllers, one or more DSPs, one or more ASICs, or one or more FPGAs, as well as other discrete or integrated logic circuitry. The functions attributed to processing circuitry 96 herein may be embodied as software, firmware, hardware or any combination thereof. In some examples, processing circuitry 96 may perform one or more techniques described herein. For example, processing circuitry 96 may determine estimates of serum potassium and estimates of GFR based on ECG information collected by IMD 10.

Server 94 may include memory 98. Memory 98 includes computer-readable instructions that, when executed by processing circuitry 96, cause IMD 10 and processing circuitry 96 to perform various functions attributed to IMD 10 and processing circuitry 96 herein. Memory 98 may include any volatile, non-volatile, magnetic, optical, or electrical media, such as RAM, ROM, NVRAM, EEPROM, flash memory, or any other digital media.

In some examples, one or more of computing devices 100A-100N (e.g., device 100A) may be a tablet or other smart device located with a clinician, by which the clinician may program, receive alerts from, and/or interrogate IMD 10. For example, the clinician may access data corresponding to serum potassium in the blood of patient 4 or estimates of GFR determined by IMD 10, external device 12, processing circuitry 14, or server 94 through device 100A, such as when patient 4 is in between clinician visits, to check on a status of a medical condition, such as heart failure. In some examples, the clinician may enter instructions for a medical intervention for patient 4 into an app in device 100A, such as based on a status of a patient condition determined by IMD 10, external device 12, processing circuitry 14, or any combination thereof, or based on other patient data known to the clinician. Device 100A then may transmit the instructions for medical intervention to another of computing devices 100A-100N (e.g., device 100B) located with patient 4 or a caregiver of patient 4. For example, such instructions for medical intervention may include an instruction to change a drug dosage, timing, or selection, to schedule a visit with the clinician, or to seek medical attention. In further examples, device 100B may generate an alert to patient 4 based on a status of a medical condition of patient 4 determined by IMD 10, which may enable patient 4 proactively to seek medical attention prior to receiving instructions for a medical intervention. In this manner, patient 4 may be empowered to take action, as needed, to address his or her medical status, which may help improve clinical outcomes for patient 4.

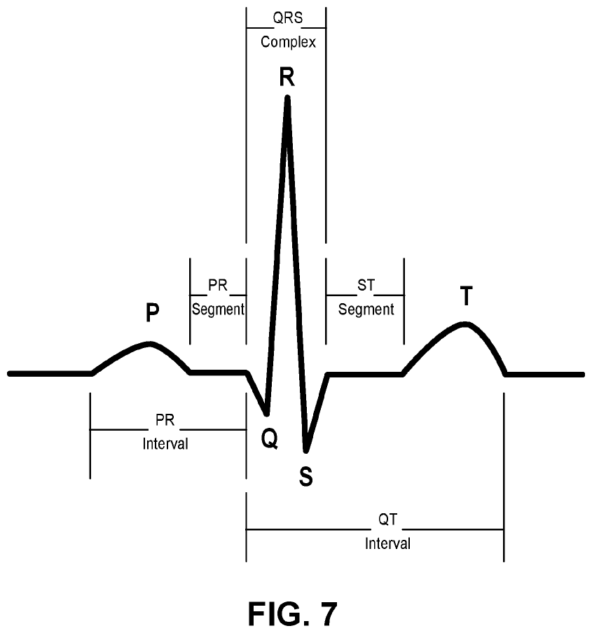

FIG. 7 is a conceptual diagram of an example portion of an ECG. There may be a correlation between ECG morphological features and serum potassium. For example, a T-wave amplitude and width, a QT interval, an ST segment, a PR interval, a P-wave amplitude, a QRS complex width, and/or an R-wave amplitude may be indicative of a level of serum potassium. Therefore, by analyzing one or more of such morphological features processing circuitry, such as processing circuitry 50, may be able to determine an estimate of serum potassium. T-wave morphology and R-wave amplitude may be the most sensitive morphological features to changes in serum potassium.

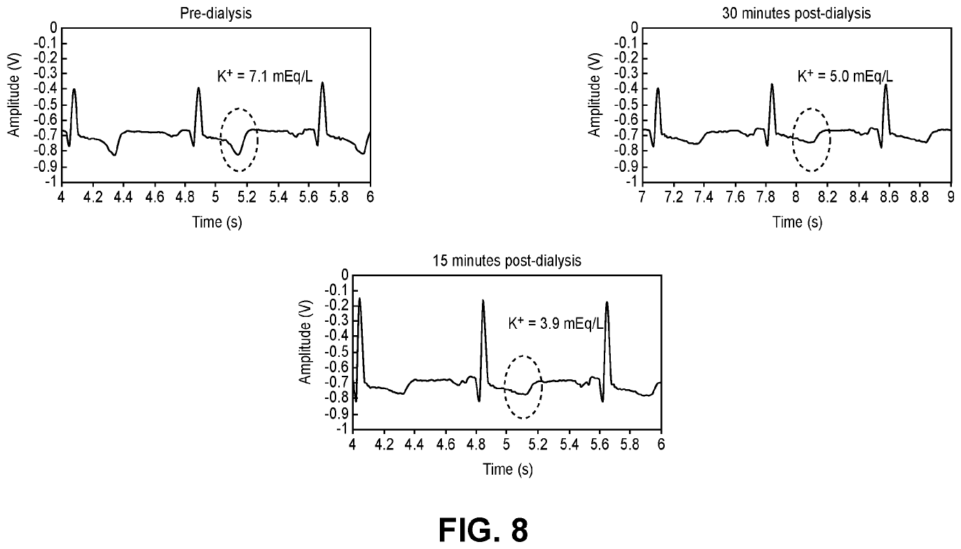

FIG. 8 is a conceptual diagram of example ECGs taken pre-dialysis and post-dialysis. Monitoring ECG data pre-dialysis and post dialysis shows changes in the morphological features of the ECG. For example, there are visually apparent morphology changes to the T-wave amplitude, the R-wave amplitude (which varies inversely with serum potassium, and ST elevation or amplitude (which are circled) at high levels of serum potassium.

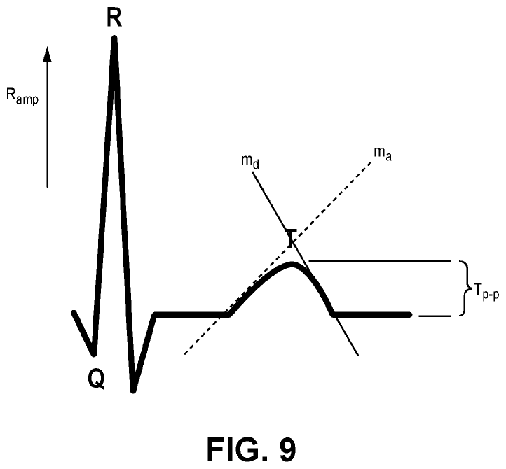

FIG. 9 is a conceptual drawing of example morphological features of an ECG which may be used to determine an estimate of serum potassium in accordance with one or more techniques described herein. The T-wave amplitude peak-to-peak (Tₚ₋ₚ) may be one such feature. This T-wave amplitude peak-to-peak may be normalized by the R-wave amplitude (Rₐₘₚ). For example, processing circuitry 50 may normalize a determined T-wave amplitude peak-to-peak by the R-wave amplitude. Furthermore, the ascending slope of the T-wave (mₐ), the descending slope of the T-wave (m_{d}) and the intersection point between the ascending slope of the T-wave and the descending slope of the T-wave may also be used to estimate serum potassium. In some examples, processing circuitry, such as processing circuitry 50, may use additional or other morphological feature(s) of the ECG to determine an estimate of serum potassium, such as a T-wave offset. In some examples, processing circuitry 50 may average the normalized T-wave morphology across several successive beats, such as over a 30 second time period or longer.

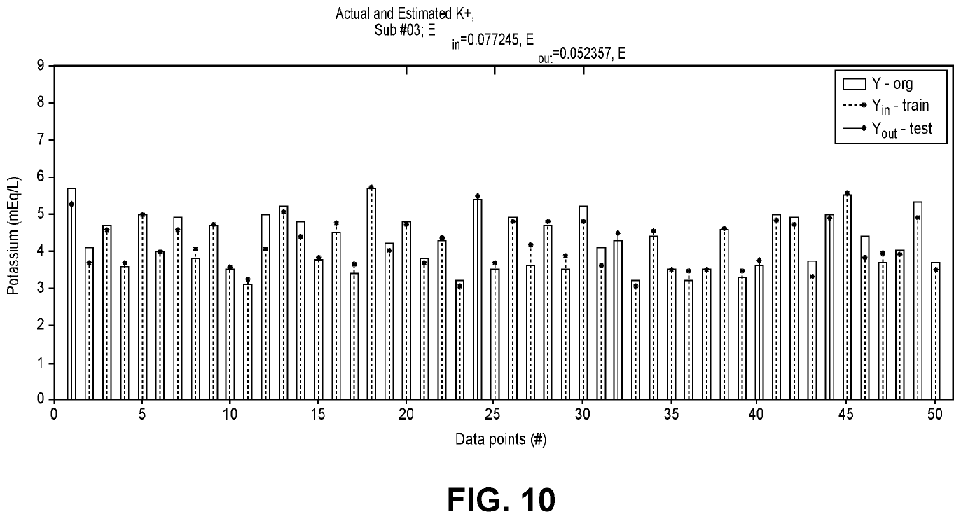

FIG. 10 is a graphical diagram of example data for a patient-specific machine learned model in accordance with one or more techniques described herein. In the example of FIG. 10, data input into the patient-specific machine learned model may include six T-Wave morphological features including T-wave amplitude, T-wave slope(s), and T-wave offset. Data was collected for a number of individual subjects in the range of 12-57 data points. A ten-fold cross validation was conducted. The average error out from the patient-specific machine learned model of 0.3. The sensitivity was 90% (within ±1.0 mEq/L).

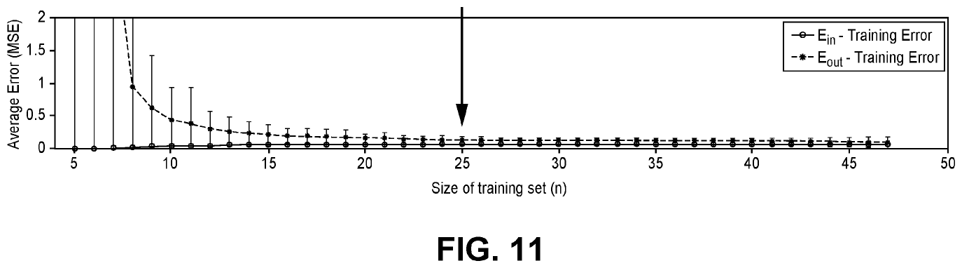

FIG. 11 is a graphical diagram of an example average error for a patient specific machine learned model per the size of a training set in accordance with one or more techniques described herein. As can be seen in the example of FIG. 11, by training with a training set of approximately 25 data points or samples, processing circuitry, such as processing circuitry 50 may be able to determine a fairly accurate estimate of serum potassium based on the ECG morphology.

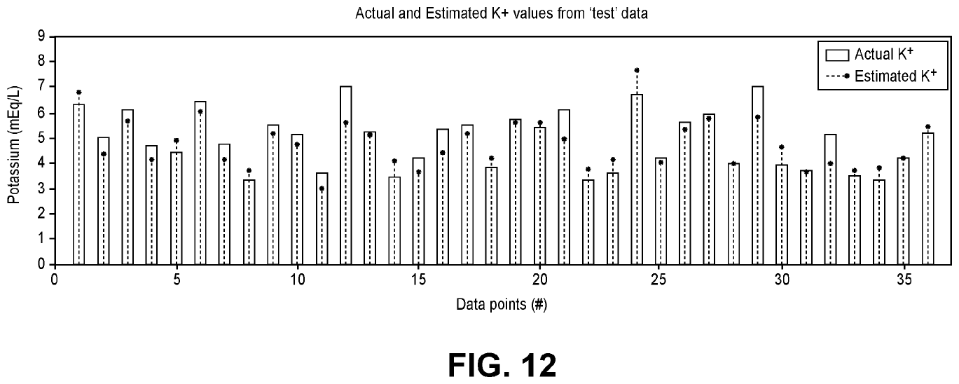

FIG. 12 is a graphical diagram of example data for a population-averaged machine learned model in accordance with one or more techniques described herein. In some examples, the population-averaged machine learned model is a linear mixed effects model. In some examples, the population-averaged machine learned model may be represented by the formula yᵢ=Xᵢβ+Zᵢbᵢ+εᵢ, where yᵢ is the nᵢ×1 response vector for observations in the ith group, Xᵢ is the nᵢ×p model matrix for the fixed effects for observations in group i, β is the p×1 vector of fixed-effects coefficients, Zᵢ is the nᵢ×q model matrix for the random effects for observations in group i, bᵢ is the q×1 vector of random-effect coefficients for group i, and εᵢ is the nᵢ×1 vector of errors for observations in group i. In the example of FIG. 12, data input into the population-averaged machine learned model may include six T-Wave morphological features, 7 fixed-effect parameters, and 7 random-effect parameters. Data was collected from 20 subjects for a total of 640 data points.

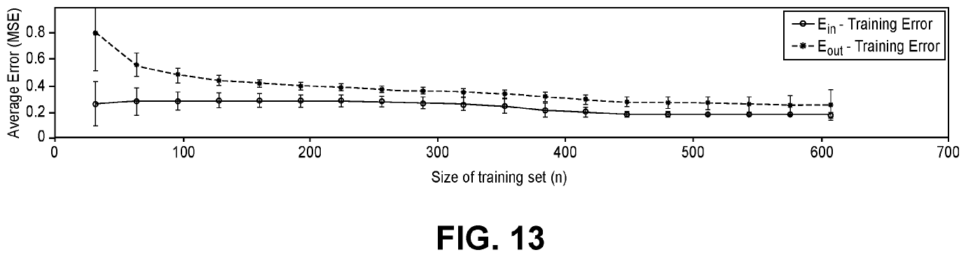

FIG. 13 is a graphical diagram of an example average error for a population-averaged machine learned model per the size of a training set in accordance with one or more techniques described herein. As can be seen, with a larger training set, the training error and testing error approach each other.

For example, serum creatine measured from blood drawn from patient 4, an age of patient 4, and a gender of patient 4 may be used to determine an estimate of GFR. A linear regression or machine learning model may be used to determine an estimate of GFR from ECG. Concurrently, IMD 10, external device 12, and/or processing circuitry 14 may monitor an ECG of patient 4 and, based on, for example, R-R intervals, determine an HRV of patient 4.

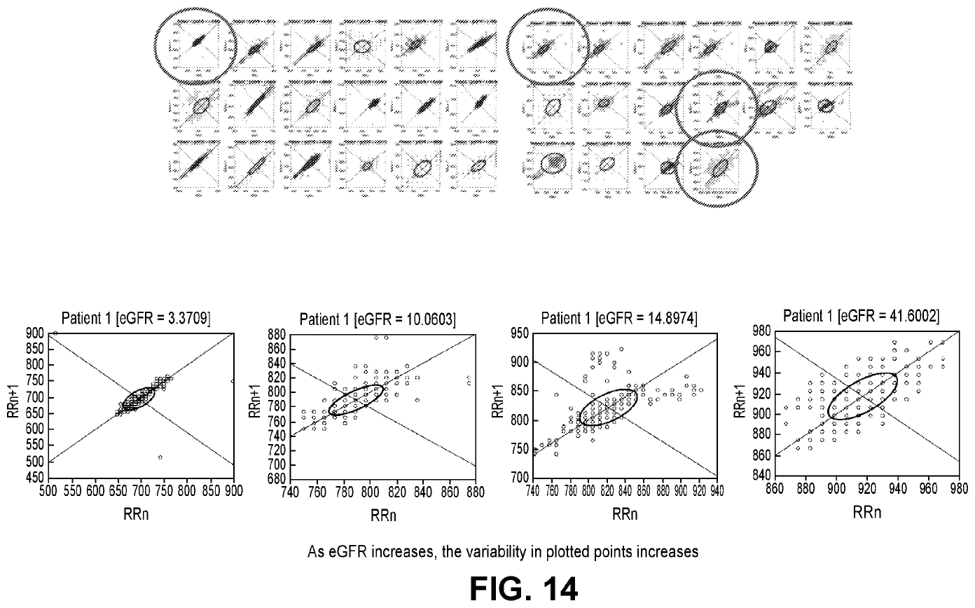

FIG. 14 is a graphical diagram of Poincaré plots in accordance with one or more techniques described herein. For example, IMD 10, external device 12, and/or processing circuitry 14 may use Poincaré plots to determine an estimate of GFR. Poincaré plots may be a geometric, nonlinear technique to assess dynamics of HRV. From the Poincaré plots, IMD 10, external device 12, and/or processing circuitry 14 may determine a standard descriptor 1, a standard descriptor 2, ratio of the standard descriptors, and/or the area of the ellipse in the Poincaré plots. Standard descriptor 1 and standard descriptor 2 may describe the shape of the Poincaré plot. This information may be used to calculate an estimate of GFR. For example, the estimate of GFR may increase as the variability of the plotted points increases. Thus, there may be a relationship between the variability of the plotted points of the Poincaré plots and the estimate of GFR.

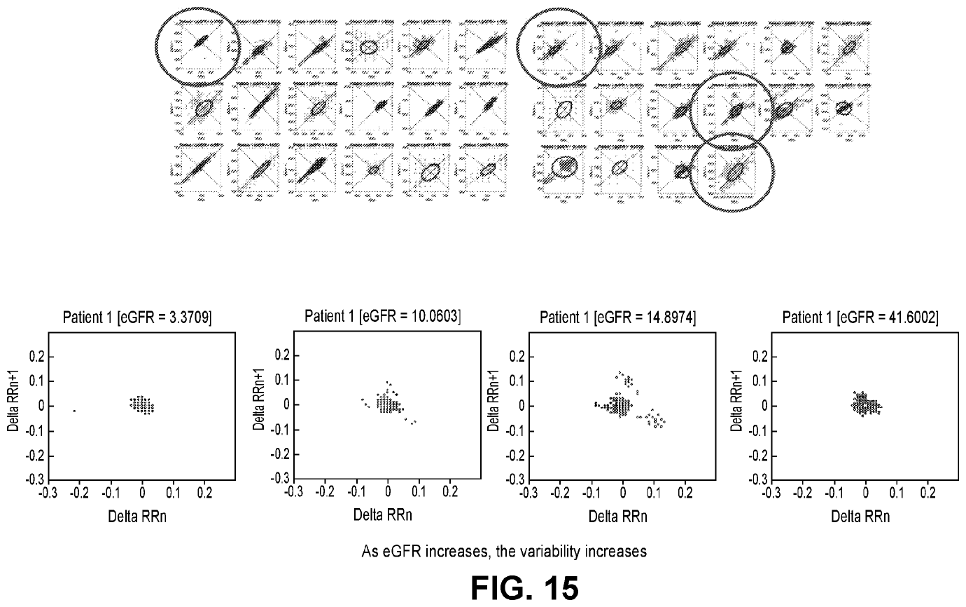

FIG. 15 is a graphical diagram of Lorenz plots in accordance with one or more techniques described herein. For example, IMD 10, external device 12, and/or processing circuitry 14 may use Lorenz plots when determining an estimate of GFR. In some examples, IMD 10, external device 12, and/or processing circuitry 14 may use a different interval than for Poincaré plots with the Lorenz plots. The different interval may provide for better visibility of variation in collected data. For example, IMD 10, external device 12, and/or processing circuitry 14 may determine a qualitative assessment of the data by determining the variability of plotted points about an origin of the Lorenz plots. Additionally, or alternatively, IMD 10, external device 12, and/or processing circuitry 14 may determine a quantitative assessment of the data by determining the sparsity and/or density of the plotted points of the Lorenz plots. In some examples, IMD 10, external device 12, and/or processing circuitry 14 may use both Lorenz plots and Poincaré plots when determining the estimate of GFR.

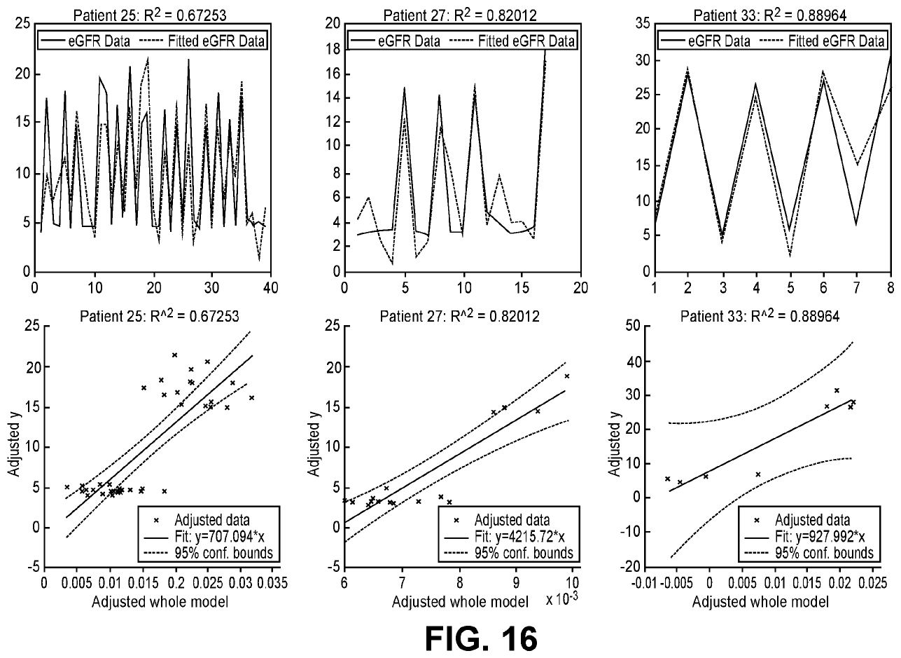

FIG. 16 is a graphical diagram illustrating examples of estimation of GFR based on HRV in an ECG in accordance with one or more techniques described herein. For example, IMD 10, external device 12, and/or processing circuitry 14 may monitor an ECG of patient 4, determine a plurality of HRV metrics, and estimate the GFR based on such metrics. For example, such HRV metrics may include: 1) a root mean square of successive differences between heartbeats (e.g., between R-waves), 2) from Poincaré plots, standard descriptors 1 and 2, a ratio of the standard descriptors 1 and 2, and/or the area of the ellipses; and/or 3) from the Lorenz plots, sparsity and/or density of the plotted points. IMD 10, external device 12, and/or processing circuitry 14 may use a linear regression or a machine learning model to determine an estimate of GFR.

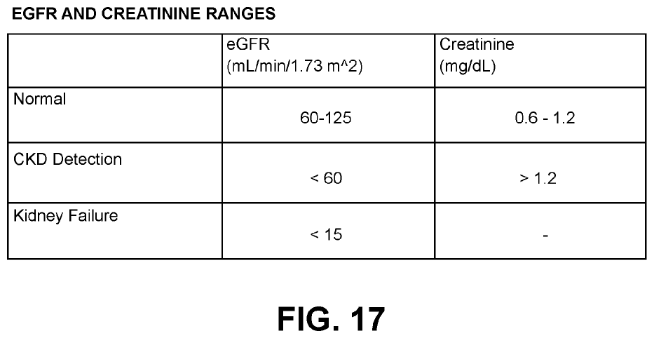

FIG. 17 is a tabular diagram illustrating an example of estimated GFR and serum creatine in accordance with one or more techniques described herein. As can be seen, there is a relationship between serum creatine levels and estimated GFR.

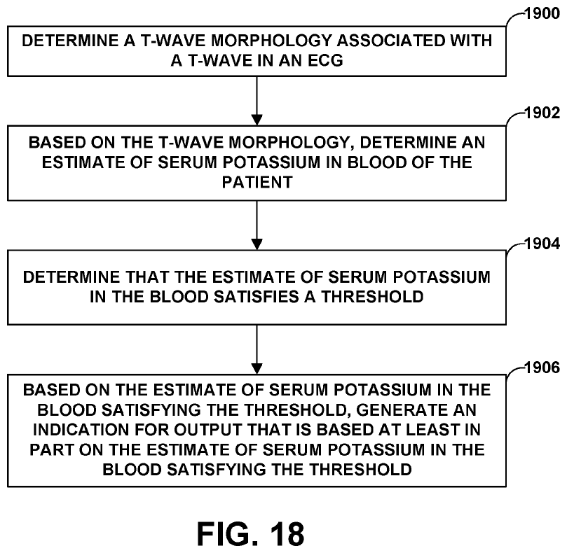

FIG. 18 is a flow diagram illustrating an example of dynamically adjusting an impedance measurement range in accordance with one or more techniques of this disclosure. The example of FIG. 19 is focused on processing circuitry 50 of IMD 10 performing one or more of the techniques of this disclosure. However, processing circuitry 14, processing circuitry 50, processing circuitry 80, processing circuitry 96, or any combination thereof, may perform one or more of the techniques of this disclosure.

Processing circuitry 50 may determine a T-wave morphology associated with a T-wave in the ECG (1900). For example, sensing circuitry 52 may sense an ECG of patient 4 and processing circuitry 50 may process the ECG to determine the T-wave morphology in the ECG. Based on the T-wave morphology, processing circuitry 50 may determine an estimate of serum potassium in blood of the patient (1902). For example, processing circuitry 50 may apply at least one of a patient specific machine learned model or a population averaged machine learned model to the morphology of the T-wave to determine the estimate of serum potassium in the blood of the patient.

Processing circuitry 50 may determine that the estimate of serum potassium in the blood satisfies a threshold (1904). For example, processing circuitry 50 may compare the estimate of serum potassium to a threshold to determine whether the estimate of serum potassium in the blood satisfies the threshold. Based on the estimate of serum potassium in the blood satisfying the threshold, processing circuitry 50 may generate an indication for output that is based at least in part on the estimate of serum potassium in the blood satisfying the threshold (1906). For example, processing circuitry 50 may generate an alert or a recommendation of remedial action, such as change a medication, change a dosage or frequency of the medication, or other remedial action.

In some examples, processing circuitry 50 may determine an R-wave morphology associated with an R-wave in the ECG, the R-wave preceding the T-wave. Processing circuitry 50 may normalize the T-wave morphology based on the R-wave morphology prior to determining the estimate of serum potassium in the blood. In some examples, processing circuitry 50 may average the normalized T-wave across several successive heart beats, such as over a 30 second time period or longer.

In some examples, based on the estimate of serum potassium in the blood satisfying the threshold, processing circuitry 50 may control the sensing circuitry to increase a sampling rate of the ECG. Processing circuitry 50 may monitor the ECG for arrythmia of a heart of the patient.

In some examples, processing circuitry 50 may determine an arrythmia of the heart of the patient. Based on determining the arrythmia of the heart of the patient, processing circuitry 50 may perform at least one of pace the heart of the patient or generate an indication of arrythmia for output.

This disclosure includes the following non-limiting examples.

### Example 1

A medical device system comprising: a plurality of electrodes; sensing circuitry configured to sense an ECG of a patient; and processing circuitry configured to: determine a T-wave morphology associated with a T-wave in the ECG; based on the T-wave morphology, determine an estimate of serum potassium in blood of the patient; determine that the estimate of serum potassium in the blood satisfies a threshold; and based on the estimate of serum potassium in the blood satisfying the threshold, generate an indication for output that is based at least in part on the estimate of serum potassium in the blood satisfying the threshold.

### Example 2

The medical device system of example 1, wherein the processing circuitry is further configured to: determine an R-wave morphology associated with an R-wave in the ECG, the R-wave preceding the T-wave; and normalize the T-wave morphology based on the R-wave morphology prior to determining the estimate of serum potassium in the blood.

### Example 3

The medical device system of example 2, wherein the processing circuitry is further configured to average the normalized T-wave morphology across a plurality of successive heart beats.

### Example 4

The medical device system of any combination of examples 1-3, wherein the indication comprises at least one of an alert or a recommendation of remedial action.

### Example 5

The medical device system of any combination of examples 1-4, wherein the processing circuitry is further configured to: based on the estimate of serum potassium in the blood satisfying the threshold, control the sensing circuitry to increase a sampling rate of the ECG; and monitor the ECG for arrythmia of a heart of the patient.

### Example 6

The medical device system of example 5, wherein the processing circuity is further configured to: determine an arrythmia of the heart of the patient; and based on determining the arrythmia of the heart of the patient, perform at least one of pace the heart of the patient or generate an indication of arrythmia for output.

### Example 7

The medical device system of any combination of examples 1-6, wherein determining the estimate of the serum potassium in the blood comprises applying at least one of a patient specific machine learned model or a population averaged machine learned model to the morphology of the T-wave.

### Example 8

A method practiced by the medical device system of any of examples 1-7.

### Example 9

A non-transitory computer-readable storage medium storing instructions, which when executed, cause processing circuitry to perform as in any of examples 1-7.

### Example 10

The medical device system of any of examples 1-7, wherein the processing circuity is further configured to: determine, based at least in part on an accelerometer signal, a posture of the patient; and determine, based at least in part on the posture of the patient, the estimate of serum potassium in blood of the patient.

### Example 11

The medical device system of any of examples 1-7 or 10, wherein the posture is supine.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as clinician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

Various examples have been described. These and other examples are within the scope of the following claims.

1. A medical device system comprising:
a plurality of electrodes;
sensing circuitry configured to sense an electrocardiogram (ECG) of a patient; and
processing circuitry configured to:
determine a T-wave morphology associated with a T-wave in the ECG;
based on the T-wave morphology, determine an estimate of serum potassium in blood of the patient;
determine that the estimate of serum potassium in the blood satisfies a threshold; and
based on the estimate of serum potassium in the blood satisfying the threshold, generate an indication for output that is based at least in part on the estimate of serum potassium in the blood satisfying the threshold.

2. The medical device system of claim 1, wherein the processing circuitry is further configured to:
determine an R-wave morphology associated with an R-wave in the ECG, the R-wave preceding the T-wave; and
normalize the T-wave morphology based on the R-wave morphology prior to determining the estimate of serum potassium in the blood.

3. The medical device system of claim 2, wherein the processing circuitry is further configured to average the normalized T-wave morphology across a plurality of successive heart beats.

4. The medical device system of claim 1, wherein the indication comprises at least one of an alert or a recommendation of remedial action.

5. The medical device system of claim 1, wherein the processing circuitry is further configured to:
based on the estimate of serum potassium in the blood satisfying the threshold, control the sensing circuitry to increase a sampling rate of the ECG; and
monitor the ECG for arrythmia of a heart of the patient.

6. The medical device system of claim 5, wherein the processing circuity is further configured to:
determine an arrythmia of the heart of the patient; and
based on determining the arrythmia of the heart of the patient, perform at least one of control the medical device system to pace the heart of the patient or generate an indication of arrythmia for output.

7. The medical device system of claim 1, wherein determining the estimate of the serum potassium in the blood comprises applying at least one of a patient specific machine learned model or a population averaged machine learned model to the morphology of the T-wave.

8. The medical device system of claim 1, wherein the processing circuity is further configured to:
determine, based at least in part on an accelerometer signal, a posture of the patient; and
determine, based at least in part on the posture of the patient, the estimate of serum potassium in blood of the patient.

9. The medical device system of any of claims 1, wherein the posture is supine.

10. A method for operating processing circuitry of a medical system comprising:
determining, by the processing circuitry, a T-wave morphology associated with a T-wave in a sensed electrocardiogram (ECG) of a patient;
determining, by the processing circuitry and based on the T-wave morphology, an estimate of serum potassium in blood of the patient;
determining, by the processing circuitry, that the estimate of serum potassium in the blood satisfies a threshold; and
generating, by the processing circuitry and based on the estimate of serum potassium in the blood satisfying the threshold, an indication for output that is based at least in part on the estimate of serum potassium in the blood satisfying the threshold.

11. (canceled)

12. The method of claim 10, wherein the processing circuitry is further configured to:
determining, by the processing circuitry, an R-wave morphology associated with an R-wave in the sensed ECG, the R-wave preceding the T-wave; and
normalizing, by the processing circuitry, the T-wave morphology based on the R-wave morphology prior to determining the estimate of serum potassium in the blood.

13. The method of claim 12, further comprising averaging the normalized T-wave morphology across a plurality of successive heart beats.

14. The method of claim 10, wherein the indication comprises at least one of an alert or a recommendation of remedial action.

15. The method of claim 10, further comprising:
controlling, by the processing circuitry and based on the estimate of serum potassium in the blood satisfying the threshold, sensing circuitry to increase a sampling rate of the ECG; and
monitoring, by the processing circuitry, the ECG for arrythmia of a heart of the patient.

16. The method of claim 15, further comprising:
determining, by the processing circuitry, an arrythmia of the heart of the patient; and
performing, by a medical device system and based on determining the arrythmia of the heart of the patient, at least one of pace the heart of the patient or generate an indication of arrythmia for output.

17. The method of claim 10, wherein determining the estimate of the serum potassium in the blood comprises applying at least one of a patient specific machine learned model or a population averaged machine learned model to the morphology of the T-wave.

18. The method of claim 10, further comprising:
determining, by the processing circuitry and based at least in part on an accelerometer signal, a posture of the patient; and
determining, by the processing circuitry and based at least in part on the posture of the patient, the estimate of serum potassium in blood of the patient.

19. The method of claim 18, wherein the posture is supine.

20. A non-transitory computer-readable storage medium storing instructions, which when executed, cause processing circuitry to:
determine a T-wave morphology associated with a T-wave in a sensed electrocardiogram (ECG) of a patient;
based on the T-wave morphology, determine an estimate of serum potassium in blood of the patient;
determine that the estimate of serum potassium in the blood satisfies a threshold; and
based on the estimate of serum potassium in the blood satisfying the threshold, generate an indication for output that is based at least in part on the estimate of serum potassium in the blood satisfying the threshold.

21. The non-transitory computer-readable storage medium of claim 20, wherein the instructions further cause the processing circuitry to:
determine an R-wave morphology associated with an R-wave in the sensed ECG, the R-wave preceding the T-wave; and
normalize the T-wave morphology based on the R-wave morphology prior to determining the estimate of serum potassium in the blood.
